(19) United States
(12) Patent Application Publication
(10) Pub. No.: US 2003/0093299 A1
(43) Pub. Date: May 15, 2003

(54) Method and system for providing medical consulting services, with automatic remuneration to the service provider

(75) Inventors: Rainer Kuth, Herzogenaurach (DE); Christoph Zindel, Uttenreuth (DE)

Correspondence Address: SCHIFF HARDIN & WAITE, 6600 SEARS TOWER, 233 S WACKER DR, CHICAGO, IL, 60606-6473, US

(73) Assignee: Siemens Aktiengesellschaft

(21) Appl. No.: 10/277,288

(22) Filed: Oct. 22, 2002

(30) Foreign Application Priority Data
Oct. 25, 2001 (DE) 10152735.7

(51) Int. Cl. G06F 17/60; A61B 5/00

(52) U.S. Cl. 705/2; 705/3; 600/300

(57) ABSTRACT

In a system for making expert knowledge accessible to a user, such as a physician, of medical examination equipment, after an inquiry from the user to a physician with specialized knowledge, a system for transmitting a handling procedure in the form of pulse sequences, protocols, sets of parameters or the like for the user's examination equipment is transmitted via a communication system to the user, from the specialized physician. The handling procedure is tailored by the specialized physician to actual medical diagnoses of the requesting user. The communication system is coupled with a license accounting system which arranges for the expert creating the handling procedure to receive a royalty.

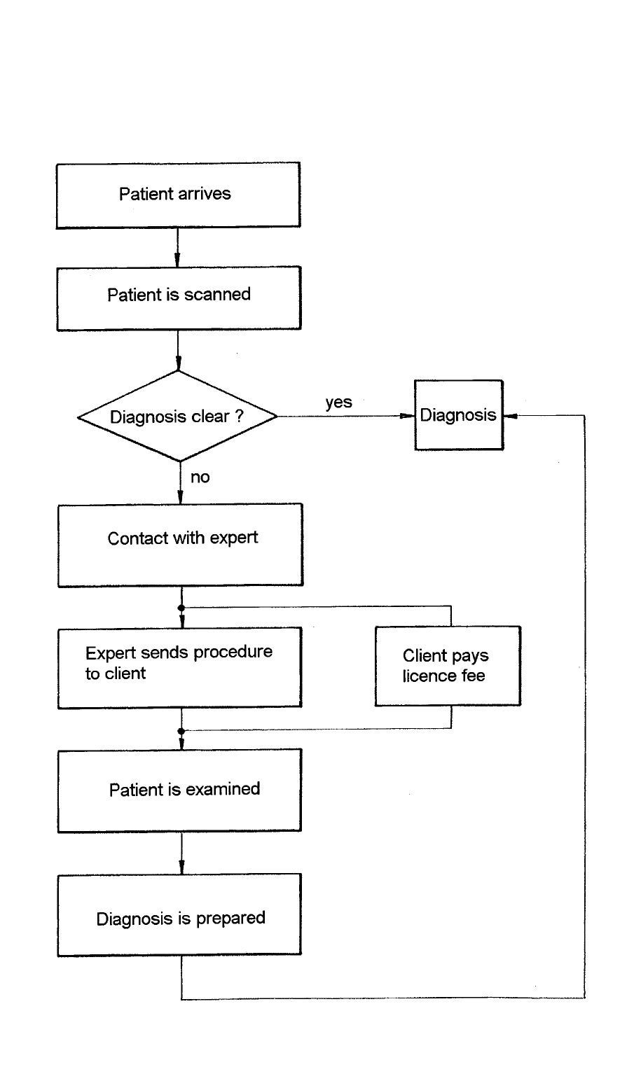

### BACKGROUND OF THE INVENTION

1. Field of the Invention

The present invention relates to a system for making expert knowledge available to a user, such as physician, of medical examination equipment.

2. Description of the Prior Art

The complexity involved in using medical examination equipment, in particular in the case of radiological examinations, is constantly increasing. An increasing number of diagnoses require profound specialist knowledge with respect to very rarely occurring disorders. Therefore, for many physicians, specifically in the case of radiologists, there is a need for expert advice and support from highly specialized skilled colleagues. The latter are familiar with clinical procedures or are able to develop them. Such procedures may be based, for example, on special pulse sequences, protocols, sets of parameters or the like which must be protected from unlicensed use to allow them to be commercially exploited.

### SUMMARY OF THE INVENTION

An object of the present invention is to provide a system for making such knowledge of specialists available to the ordinary skilled colleagues, while also ensuring that the specialists are reimbursed in a suitable way.

This object is achieved according to the invention that a system for transmitting a handling procedure in the form of pulse sequences, protocols, sets of parameters or the like for the examination equipment, tailored to actual medical diagnoses of the requesting user, that is coupled to a license accounting system which arranges for the expert creating the handling procedure to receive a royalty, preferably for a direct debiting system which collects the royalty directly when the procedure is sent to be provided.

To allow accounting disputes—regardless of whether direct debiting or regular billing is used—to be avoided as far as possible, or settled unproblemmatically, in a embodiment of the invention the procedure is acquired by the expert providing it. The “expert” may be a physician with specialized practice or a highly specialized expert system. The user can receive the procedure, with the date, time, name of the user, name of the patient and possibly the cost for the license fee.

There is a risk that the expert (if a person) may not be motivated to participate in the system if there is a risk of the user paying a royalty for using a procedure once and then being able to continue using the procedure made available to him repeatedly without any payment. Therefore in an embodiment of the invention the handling procedure contains a starting time and/or an expiration time, before and after which it cannot be used. It also is possible for the handling procedure to contain a user limitation to a predeterminable number of uses.

In a further embodiment of the invention, the user limitation may be in the form of an identification code contained in the handle procedure. which restricts use to one or more target systems and/or specifically defined patients or probands. With the permanent link with the name of the patient for whom the procedure has been created or selected by the expert, the user, that is the ordinary skilled colleague in situ, can only use it in his or her examination equipment for this one patient. This user limitation may in this case be further simplified by the handling procedure being encoded as a set of data which can be fed directly into the user's examination equipment to control its parameter settings and examination sequences.

When using the system, the user, in particular a radiologist, consults the expert or a highly specialized expert system on account of an unclear diagnosis, possibly submitting the existing radiological findings, whereupon the expert selects or newly creates a suitable handling procedure for carrying out further specially configured examinations with the user's examination equipment and transmits this handling procedure to the user via the transmission system accompanied by simultaneous accounting procedures for the license fee.

### DESCRIPTION OF THE DRAWING

The single FIGURE is a flowchart illustrating the basic steps of the inventive method, as practiced by the inventive system.

### DESCRIPTION OF THE PREFERRED EMBODIMENTS

After arriving at the premises of the user, for example a radiologist, a patient is scanned on the basis of an initial suspicion arising from a preliminary examination, conducted either by the radiologist or a family physician who has referred the patient. If, thereafter that, the diagnosis is clear, the procedure has been completed.

If, however, the diagnosis is unclear or the radiologist cannot carry out any suitable examination on the basis of the initial suspicion of the family doctor, the consultancy system under license-controlled conditions according to the invention comes into play. For this purpose, the user contacts an expert, in particular a specialized skilled colleague at a university, and presents him or her with the initial diagnosis, possibly transmitting the existing radiological examination findings. In response to this, the expert sends a handling procedure to the client, containing the necessary examination steps while at the same time specifying the optimum setting of the equipment parameters for this special examination, which can best take place in the form of a set of data which can be fed directly into the control system of the examination equipment. Sending of this handling procedure is accompanied at the same time by billing for the royalty to be paid, in particular the direct debiting of this royalty, from an account of the user set up for this purpose, to which the expert has access via the system.

The patient is then examined by the user with the medical examination equipment of the user, employing the handling procedures of the expert, and then the diagnosis is prepared.

For the rare case in which even there are still problems with the preparation of a clear diagnosis, it is of course possible, in a loop, for contact to be made once again with the expert. The expert then can, using the examination findings now available, modify or add to his or her handling procedure and send a revised version it once again to the user.

Although modifications and changes may be suggested by those skilled in the art, it is the intention of the inventors to embody within the patent warranted hereon all changes and modifications as reasonably and properly come within the scope of their contribution to the art.

We claim as our invention:

1. A system for making expert knowledge accessible to a user of medical examination equipment comprising:
an electronic communication system for, in response to an inquiry from a user including diagnostic information, transmitting a handling procedure to the user tailored by an expert to said diagnostic information; and
an electronic accounting system electronically coupled to said electronic communication system for, upon transmittal of said handling procedure, automatically arranging for remuneration to said expert for providing said handling procedure.

2. A system as claimed in claim 1 wherein said electronic communication system allows transmittal of a handling procedure selected from the group consisting of pulse sequences for said equipment, protocols for use of said equipment, and sets of parameters for operating said equipment.

3. A system as claimed in claim 1 wherein said electronic communication system also includes an archiving system which associates a date, a time, an identification of said user, and an identification of a patient, with said handling procedure.

4. A system as claimed in claim 3 wherein said archiving system also associates a cost for said remuneration with said handling procedure.

5. A system as claimed in claim 1 wherein said electronic communication system transmits said handling procedure with at least one time limitation selected from the group consisting of a starting time before which said user cannot use said handling procedure and an expiration time after which said user cannot use said handling procedure.

6. A system as claimed in claim 1 wherein said electronic communication system automatically associates a user limitation with said handling procedure limiting use of said handling procedure by said user to a predetermined number of uses.

7. A system as claimed in claim 1 wherein said electronic communication system automatically associates an identification code with said handling procedure selected from the group consisting of a restriction to use of said handling procedure with specified equipment and a restriction to use of said procedure for a specified patient.

8. A system as claimed in claim 1 wherein said electronic accounting system is a direct debiting system which electronically debits an account for said remuneration directly at a time when said handling procedure is transmitted to said user.

9. A system as claimed in claim 1 wherein said electronic communication system automatically encodes said handling procedure as a set of data adapted to be directly fed into said examination equipment to control at least one of parameter settings and examination sequences of said examination equipment.

10. A method for making expert knowledge accessible to a user of medical examination equipment comprising the steps of:
via an electronic communication system, transmitting an inquiry, containing diagnostic information, from a user of medical equipment to an expert;
said expert formulating a handling procedure tailored to said diagnostic information;
via said electronic communication system, for transmitting said handling procedure to the user; and
electronically coupling an electronic accounting system to said electronic communication system and, upon transmittal of said handling procedure, automatically arranging for remuneration to said expert for providing said handling procedure.

11. A method as claimed in claim 10 comprising transmitting, as said handling procedure, a handling procedure selected from the group consisting of pulse sequences for said equipment, protocols for use of said equipment, and sets of parameters for operating said equipment.

12. A method as claimed in claim 10 comprising, in an archiving system included in said electronic communication system associating a date, a time, an identification of said user, and an identification of a patient, with said handling procedure.

13. A method as claimed in claim 12 comprising in said archiving system, also associating a cost for said remuneration with said handling procedure.

14. A method as claimed in claim 10 comprising transmitting said handling procedure with at least one time limitation selected from the group consisting of a starting time before which said user cannot use said handling procedure and an expiration time after which said user cannot use said handling procedure.

15. A method as claimed in claim 10 comprising in said electronic communication system, automatically associates a user limitation with said handling procedure limiting use of said handling procedure by said user to a predetermined number of uses.

16. A method as claimed in claim 10 comprising in said electronic communication system, automatically associating an identification code with said handling procedure selected from the group consisting of a restriction to use of said handling procedure with specified equipment and a restriction to use of said procedure for a specified patient.

17. A method as claimed in claim 10 comprising automatically electronically debiting an account for said remuneration directly at a time when said handling procedure is transmitted to said user.

18. A method as claimed in claim 10 comprising in said electronic communication system, automatically encoding said handling procedure as a set of data adapted to be directly fed into said examination equipment to control at least one of parameter settings and examination sequences of said examination equipment.
